(19) United States
(12) Patent Application Publication
(10) Pub. No.: US 2003/0069455 A1
(43) Pub. Date: Apr. 10, 2003

(54) Method for making a substantially pure mixture of cyclododecanediol isomers and uses thereof

(76) Inventors: Samuel Livingston Lane, Kennett Square, PA (US); Edward Francis Moran JR., Gibbstown, NJ (US); John Joseph Ostermaier, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/244,222

(22) Filed: Sep. 16, 2002

Related U.S. Application Data
(60) Provisional application No. 60/324,863, filed on Sep. 26, 2001.

(51) Int. Cl. C07C 31/18; C07C 27/02

(52) U.S. Cl. 568/852; 568/876; 568/877

(57) ABSTRACT

Disclosed is a method for preparing a substantially pure mixture of cyclododecanediol isomers. The isomers include 1,4-cyclododecanediol, 1,5-cyclododecanediol, and 1,6-cyclododecanediol. Also disclosed is a method for using a substantially pure mixture of cyclododecanediol isomers, which include the preparation of diamines; esters; diesters, such as diacetates and diacrylates; bis-phenol cyclododecane; and cyclododecanediol-based polyester polymers and polyester polyols.

### FIELD OF THE INVENTION

The present invention relates to a method for preparing a substantially pure mixture of cyclododecanediol isomers, the use of these isomers.

### BACKGROUND OF THE INVENTION

There are cyclododecanediol isomers that are known. 1,4-cyclododecanediol is reported as CAS 41417-03-2, 1,5-cyclododecanediol as CAS 13474-05-0, and 1,6-cyclododecanediol as is CAS 14435-21-3. These chemicals are a member of the diol chemical family which includes hexanediol; neopentyl glycol; butanediol; propanediol (propylene glycol); ethanediol (ethylene glycol); cyclohexanedimethanol and hydrogenated bis-phenol A among others. Diols are commercially important chemicals with cumulative global production of billions of pounds annually. Cyclohexanedimethanol and hydrogenated bis-phenol A are both cycloaliphatic diols and are closely related structurally to cyclododecanediols. Despite the structural similarities between these diols and cyclododecanediols, the production of cyclododecanediols, in pure form, or on a useful scale, has not been reported. Also, a method for preparing a substantially pure mixture of 1,4-, 1,5-, and 1,6-cyclododecanediol isomers has not been reported.

### SUMMARY OF THE INVENTION

Disclosed herein is a method for preparing a substantially pure mixture of cyclododecanediol isomers, comprising: (i) contacting in a vessel cyclododecane and an oxygen-containing gas, in the presence of a catalyst, to obtain a mixture, said mixture comprising cyclododecanediol isomers; (ii) optionally, isolating a mixture of cyclododecanol and cyclododecanone; (iii) distilling the mixture obtained at a temperature of about 230° C. or less, to obtain a substantially pure mixture of cyclododecanediol isomers.

### DETAILED DESCRIPTION OF THE INVENTION

Cyclododecanediols are cycloaliphatic di-alcohols, which are useful chemical intermediates for a variety of applications. They can be used as intermediates for the production of many materials, including polyesters; copolyesters; polyester polyols, which are key intermediates for polyurethanes; adhesives; sealants; elastomers; powder coatings; liquid coatings; printing inks; dyes; and pharmaceuticals. Cyclododecanediols also can be readily converted to other useful intermediates such as diamines, which are intermediates used for preparing polyamides and polyurethanes, and diacrylates, which are intermediates used for producing acrylics. They also have use as surfactants, polymer crosslinkers, and plasticizers.

The present invention discloses a method for preparing a substantially pure mixture of cyclododecanediol isomers. Hereinafter, use of the term “CDDD” is used to represent “cyclododecanediol”. The isomers of the substantially pure mixture comprise 1,4-CDDD, 1,5-CDDD and 1,6-CDDD. By “substantially pure mixture” we mean a combination of CDDD isomers that has a purity of about 90% or greater. Also disclosed is a method of using a substantially pure mixture of cyclododecanediol isomers.

The method for preparing the mixture can be described in steps, the first of which involves contacting cyclododecane (CDD) and an oxygen-containing gas in the presence of a suitable catalyst. Examples of such catalysts include boron compounds including boric acid, cobalt octoate, cobalt naphthenate, cobalt laurate, chromium octoate, chromium naphthenate, chromium laurate, and chromium ethylhexanoate. Boron compounds or cobalt or chromium salts of fatty acids which are soluble in hydrocarbon solvents are particularly preferred. The catalyst may also be heterogeneous (i.e., insoluble). The cobalt or chromium may be contained on an ion exchange resin. Examples of these include sulfonic acid resins such as Amberlyst™ 15, Amberlyst™ XN-100, Dowex™ M-31, and Dowex™ M-31, and Dowex™ DR-2030. Amberlyst is a trademark of Rohm and Haas. Dowex is a trademark of Dow. Other catalysts that may be used are catalysts that may be used in any process for making cyclododecanol (CDDA) or cyclododecanone (CDDK).

The first step yields a mixture comprising 1,4-CDDD, 1,5-CDDD and 1,6-CDDD. The products obtained from step (i) may also include CDDA, CDDK, hydroxy cyclododecanone (HCDDK), cyclododecanedione, unreacted CDD, and 1,11-undecanediol.

Several other steps may be employed after the first step to increase the percent yield of CDDD. One option is to recycle unreacted CDD obtained from the first step so that it undergoes step (i) again. This is done by providing the unreacted cyclododecane obtained from step (i) and repeating step (i).

Another optional step is to separate CDDA and CDDK from the product obtained from step (i). This can be done by distillation or other suitable separation means.

Still, another option is a chemical reduction of the ketone functional groups to the corresponding alcohol groups. This results in the conversion of HCDDK and cyclododecanedione to useable CDDD product, and the conversion of CDDK to CDDA. The reduction is carried out using standard reduction conditions in the presence of reducing agents, or a suitable hydrogenation catalyst in the presence of hydrogen gas. The chemical reduction of HCDDK and cyclododecanedione to CDDD results in an increased overall yield to the desired CDDD product. In addition, reduction of the concentration of HCDDK, which is a close boiling component to the CDDD product, also results in energy savings and ease of operation during the distillation step to yield CDDD.

The next step in the method for preparing the substantially pure mixture is isolating the CDDD isomers. This is done by heating the product obtained in step (i), and the products of the optional steps, if employed, under distillation, at a temperature between from about 150° C. to about 230° C. and isolating the substantially pure mixture. The distillation is done, preferably, using a packed distillation column. Preferably, the pressure is below about 1 psig. The distillation can be completed batchwise or continuously.

Upon distillation of the CDDD isomers, before the substantially pure mixture of CDDD isomers is obtained, a fraction comprising a C11 straight chain aliphatic diol may be collected from the distillation. The CDDD isomers can be obtained by collecting the higher boiling fraction.

The distillation conditions for isolating the CDDD isomers should be in the ranges described herein to avoid less pure yields, or the degradation of the CDDD isomers to cyclododecenes and/or cyclododecenol.

The substantially pure mixture of CDDD isomers can be used to prepare other chemical materials that are useful as intermediates in a host of applications. For example, the CDDD isomers can be contacted with ammonia to prepare diamines, which in turn are useful for preparing polyamides or polyurethanes. Also, CDDD isomers can be contacted with carboxylic acids to prepare corresponding esters. Examples include, but are not limited to, the use of acrylic acid to prepare diacrylates (which, in turn, are useful for preparing acrylic ester polymers and copolymers) and the use of acetic acid to prepare diacetates.

The following examples are provided, but are not intended to limit the invention in any way.

### EXAMPLES

### Comparative Example A

### Distillation Using a Packed Column at 60-100 mm Hg Pressure Resulting in a High Distillation Vessel Temperature to Demonstrate the Decomposition of Cyclododecanediols to Cyclododecenol

Approximately 4,520 grams of the higher boiling fraction from the distillation of cyclododecanol and cyclododecanone from the oxidation product of cyclododecane were placed in a 5,000-ml distillation flask. The fraction analyzed: 6.0% cyclododecanol, 75.4% cyclododecanediol, 2.0% 1.11-undecanediol and 3.4% hydroxy cyclododecanone. The fraction was distilled through a 15-inch high, 2-inch diameter packed column. Monel 0.24×0.24-inch Pro-Pack distillation packing was used in the column. The column was topped with an automatic liquid dividing distilling head and a 300-mm Double Cooling condenser. The distillation was done under vacuum in the range of 60-70 mm Hg. The results are shown in Table-A and indicate a high degree of decomposition of cyclododecanediol to cyclododecenol with a distillation flask temperature above 230° C.

TABLE A

| | | % Cyclodo- | % | % | |
|---|---|---|---|---|---|
| | Vessel | decanol and | Cyclodo- | 1,11- | % Hydroxy |
| Percent | Temp. | Cyclodo- | decane | Undecane | Cyclodo- |
| Distilled | (° C.) | deceneol | diols | diol | decanone |
| 4.2⁽¹⁾ | 235 | 51.8 | 22.6 | 2.8 | 2.8 |
| 19.5 | 197 | 17.2 | 59.1 | 3.1 | 5.0 |
| 36.3 | 207 | 3.8 | 81.4 | 2.7 | 4.6 |
| 43.2 | 211 | 1.5 | 86.6 | 2.2 | 4.0 |
| 57.5 | 214 | 0.76 | 89.1 | 1.8 | 3.4 |
| 72.6 | 220 | 0.41 | 91.3 | 1.1 | 2.3 |
| 77.5 | 242 | 1.7 | 89.1 | 1.0 | 1.7 |
| 79.2 | 252 | 4.5 | 79.6 | 1.8 | 1.1 |
| 82.5 | 274 | 4.9 | 70.2 | 0.95 | 0.61 |

⁽¹⁾Pressure in the range of 90-100 mm Hg.

### Example 1

### Distillation Using a Packed Column at 8 mm Hg Pressure Low Temperature of Distillation Vessel Demonstrates no Decomposition of Cyclododecanediols to Cyclododecenol; and Good Separation

Approximately 2,260 grams of the higher boiling fraction from the distillation of cyclododecanol and cyclododecanone from the oxidation product of cyclododecane were placed in a 3,000-ml distillation flask. The fraction analyzed: 6.2% cyclododecanol, 76.6% cyclododecanediol, 1.8% 1,11-undecanediol and 2.8% hydroxy cyclododecanone. The fraction was distilled through a 15-inch high, 2-inch diameter packed column. Monel 0.24×0.24-inch Pro-Pack distillation packing was used in the column.

The column was topped with an automatic liquid dividing distilling head and an Allihn Type condenser. The vacuum distillation was done at 6-8 mm Hg. The results are shown in Table-1 and indicate essentially no decomposition to cyclododecenol, and the separation of the cyclododecanediol in high purity.

TABLE 1

| | | % Cyclodo- | % | % | |
|---|---|---|---|---|---|
| | Vessel | decanol and | Cyclodo- | 1, 11- | % Hydroxy |
| Percent | Temp. | Cyclodo- | decane | Undecane | Cyclodo- |
| Distilled | (° C.) | deceneol | diols | diol | decanone |
| 7.4 | 166 | 56.1 | 0.7 | 0.3 | 0.00 |
| 21.4 | 179 | 35.5 | 11.1 | 1.7 | 4.1 |
| 24.3 | 186 | 5.6 | 43.8 | 6.6 | 13.6 |
| 29.0 | 187 | 3.1 | 52.9 | 7.9 | 16.0 |
| 31.3 | 186 | 1.0 | 70.6 | 8.2 | 14.1 |
| 40.8 | 198 | 0.05 | 90.6 | 3.4 | 4.1 |
| 62.2 | 198 | 0.00 | 95.8 | 1.6 | 1.7 |
| 94.7 | 211 | 0.00 | 96.9 | 0.80 | 0.93 |

### Example 2

### Distillation Using a Packed Column at 8 mm Hg Pressure and Low Temperature in Distillation Vessel. Demonstrates no Decomposition of Cyclododecanediols to Cyclododecenol, and Good Separation

Approximately 706 grams of a higher boiling fraction from the distillation of cyclododecanol and cyclododecanone from the oxidation product of cyclododecane were placed in a 1,000-ml distillation flask. The fraction analyzed: 6.7% cyclododecanol, 72.5% cyclododecanediol, 2.0% 1,11-undecanediol and 3.1% hydroxy cyclododecanone. The fraction was distilled through a 15-inch high, 2-inch diameter packed column. Monel 0.24×0.24-inch Pro-Pack distillation packing was used in the column. The column was topped with simple distilling head with a thermometer well. The vacuum distillation was done at 8 mm Hg. The results shown in Table-2 indicate essentially no decomposition to cyclododecenol and the separation of the cyclododecanediol in high purity.

TABLE 2

| | | % Cyclodo- | % | % | |
|---|---|---|---|---|---|
| | Vessel | decanol and | Cyclodo- | 1,11- | % Hydroxy |
| Percent | Temp. | Cyclodo- | decane | Undecane | Cyclodo- |
| Distilled | (° C.) | decenol | diols | diol | decanone |
| 9.2 | 175 | 81.8 | 4.2 | 0.24 | 0.79 |
| 19.5 | 181 | 6.0 | 35.0 | 6.8 | 16.2 |
| 31.4 | 186 | 0.33 | 84.1 | 5.5 | 6.8 |
| 42.1 | 187 | 0.15 | 93.7 | 2.1 | 2.2 |
| 69.2 | 186 | 0.07 | 97.2 | 0.52 | 0.44 |
| 72.6 | 191 | 0.29 | 97.3 | 0.17 | 0.10 |
| 74.3 | 194 | 0.56 | 96.7 | 0.22 | 0.11 |

### Example 3

### Distillation Using a Packed Column at 8 mm Hg Pressure Using a Low Temperature Distillation Vessel. Demonstrates no Decomposition of Cyclododecanediols to Cyclododecenol, and Good Separation

Approximately 3,900 grams of semi-refined the higher boiling fraction from the distillation of cyclododecanol and cyclododecanone from the oxidation product of cyclododecane were placed in a 5,000-ml distillation flask. The charge to the distillation flask was not analyzed. The fraction was distilled through a 15-inch high, 2-inch diameter packed column. Monel 0.24×0.24-inch Pro-Pack distillation packing was used in the column. The column was topped with an automatic liquid dividing distilling head and a 300-mm Double Cooling Condenser. The vacuum distillation was done at 8 mm Hg. The results are shown in Table-3 and indicate essentially no decomposition to cyclododecenol and the separation of the cyclododecanediol in high purity.

TABLE 3

| | | % Cyclodo- | % | % | |
|---|---|---|---|---|---|
| | Vessel | decanol and | Cyclodo- | 1,11- | % Hydroxy |
| Percent | Temp. | Cyclodo- | decane | Undecane | Cyclodo- |
| Distilled | (° C.) | decenol | diols | diol | decanone |
| 12.5 | 198 | 8.6 | 68.2 | 3.5 | 5.3 |
| 29.7 | 199 | 0.72 | 87.6 | 2.6 | 4.5 |
| 32.1 | 199 | 2.1 | 63.3 | 6.7 | 12.7 |
| 48.8 | 204 | 0.25 | 89.8 | 2.5 | 4.6 |
| 68.0 | 206 | 0.04 | 94.4 | 1.3 | 2.6 |
| 71.1 | 220 | 0.24 | 94.4 | 1.4 | 1.8 |
| 86.1 | 221 | 0.00 | 96.9 | 0.34 | 0.80 |
| 94.1 | 207 | 0.00 | 97.3 | 0.14 | 0.48 |

### Example 4

### Continuous Distillation of CDDD Using a Packed Column at 8 mm Hg Pressure and Low Temperature

Approximately 30 gallons of a higher boiling fraction from the distillation of cyclododecanol and cyclododecanone from the oxidation product of cyclododecane were distilled continuously. The fraction analyzed 8.6% cyclododecanol, 63% cyclododecanediol, 2.3% 1,11-undecanediol and 3.1% hydroxy cyclododecanone. The still was 4″ in diameter by 15′ long and was packed with Sulzer BX packing, and the reboiler was a 2 liter thermosyphon-type. The column was operated at 8 mm Hg top pressure and a reflux ratio of 10 to 1, which gave a 180° C. top temperature and a 220° C. bottom temperature. The column was fed continuously at a rate of 60 cc/min, and the fraction having a lower boiling point than the CDDD was taken overhead as distillate at a rate of 15 cc/min, while the CDDD and the higher boiling components were taken from the bottom of the column at a rate of 45 cc/min. The bottom fraction contained 0% cyclododecanol, 76% cyclodecanediol, 0.26% 1,11-undecanediol, 0.35% hydroxy cyclododecanone. The bottoms were then continuously distilled again to recover the cyclododecanediol as distillate and remove the high boiling fraction as tails. The column was operated at 8 mm Hg head pressure, a reflux ratio of 0.5 to 1, and a feed rate of 60 cc/min, which gave a top temperature of 200° C. and a bottoms temperature of 225° C. The CDDD distillate product was taken overhead at a rate of 30 cc/min, while the higher boiling components were taken from the bottom of the column at a rate of 30 cc/min. The CDDD distillate product contained 0.3% cyclododecanol, 96.4% cyclodecanediol, 0.49% 1,11-undecanediol, 0.71% hydroxy cyclododecanone. This example illustrates that high purity CDDD can be recovered by continuous distillation.

### Example 5

### Hydrogen Reduction of the Higher Boiling Fraction to Reduce Hydroxy Cyclododecanone, an Undesirable Contaminant, to Cyclododecanediol. Demonstrates How to Increase the Concentration of the Desired Product, a Pure Mixture of Cyclododecanediol Isomers.

A pressure vessel was charged with 40.05 grams of the higher boiling fraction from the distillation of cyclododecanol and cyclododecanone from the oxidation product of cyclododecane. The fraction analyzed 8.7% cyclododecanol, 75.2% cyclododecanediol, 1.7% 1,11-undecanediol and 3.3% hydroxy cyclododecanone. 2.5 grams of commercial hydrogenation catalyst containing 5% Ruthenium on a carbon support (Engelhard Corp. ESCAT 440) was also added to the pressure vessel. The content of the vessel was heated at 125° C. under 525 psi of hydrogen for 8 hrs. It was necessary to add additional hydrogen periodically to maintain 525-psi pressure. At the end of the experiment the pressure vessel was cooled, the contents removed and the catalyst separated from the mixture. Analysis of the product indicated it contained 9.8% cyclododecanol 76.4 cyclododecanediol, 1.9% 1,11-undecanediol and 0.24 hydroxy cyclododecanone. This example demonstrates the removal of hydroxy cyclododecanone.

### Example 6

### Preparation of Cyclododecane Di-Acrylate

The formation of cyclododecane di-acrylate mixed isomers was demonstrated. The reaction of 1.5 equivalents of acrylic acid with cyclododecanediol mixed isomers at 100° C./8 hrs in the presence of a polymerization inhibitor produced a mixture containing 55-60% cyclododecane di-acrylate mixed isomers. These compounds have further use in the production of new acrylic ester polymers or can be blended with other acrylate monomers to produce co-polymers.

### Example 7A

### Preparation of Cyclododecane Esters

The formation of cyclododecane di-acetate mixed isomers and dipropionate mixed isomers was demonstrated. The reaction of a 50/50 mixture of acetic and acetic anhydride with cyclododecanediol mixed isomers at 126° C./4 hrs produced a mixture containing 72% di-acetate mixed isomers. Distillation of the reaction product produced a fraction containing 98.2% di-acetate. Likewise the reaction of the diols with propionic acid at 145° C./4 hrs produced a mixture containing 50% mixed di-propionate isomers. These compounds have further use as specialty chemical intermediates and solvents.

### Example 7B

### Preparation of Cyclododecane Diesters

Diesters of cyclododecanediol which are useful as synthetic lubricants can be prepared by reacting the cyclododecanediol isomers with an excess of monocarboxylic acids. The catalyst employed would be one of the commercially available homogeneous or heterogeneous acid esterification catalysts, for example, para-toluene Sulfonic Acid, Methane Sulfonic Acid, and Sulphonic Acid resins. Appropriate, commercially available monocarboxylic acids would be ones having 4 to 30 carbon atoms and include, but are not limited to, Valeric Acid, Isopentanoic Acid, Hexanoic Acid, Heptanoic Acid, Octanoic Acid, Isooctanoic Acid, 2-Ethylhexanoic Acid, Pelargonic Acid, Isononanoic Acid, Decanoic Acid, Neodecanoic Acid, Undecanoic Acid, Dodecanoic Acid, and Tridecanoic Acid, and mixtures thereof. These diesters are also useful as plasticizers, emollients, and solvents

### Example 8

### Preparation of Cyclododecane Diamines

The formation of mixed cyclododecane diamine isomers was demonstrated in a reaction of the mixed cyclododecanediol isomers with ammonia and hydrogen in the presence of a nickel catalyst at 190° C. and 2,400 psi. The product comprised 35% diamine. Diamines are useful as polymer intermediates, and are particularly useful when polymerized with dibasic acids to form polyamides (nylons). The diamines of cyclododecane could be used to produce a new polyamide or introduced into known polyamides to produce co-polymers.

### Example 9

### Preparation of Bis-Phenol Cyclododecane

The formation of mixed isomers of bis-phenol cyclododecane was demonstrated in a reaction of mixed cyclododecanediol isomers with excess phenol in the presence of para-toluenesulfonic acid (as an acid catalyst) at 195° C. Bis-phenol cyclododecane has a wide variety of uses in applications which presently employ bis-phenol A, imparting improved properties in many cases. These applications include, epoxy, polycarbonate polyester and vinyl ester resins.

### Example 10

### Preparation of Polyesters and Polyester Polyols

Cyclododecanediol isomers can also be used to prepare polyesters polymers and polyester polyols. To accomplish that, one would react the cyclododecanediol isomers with dibasic acids. The catalyst employed would be one of the commercially available homogeneous or heterogeneous acid esterification catalysts. Examples of these include, but are not limited to, para-toluene sulfonic acid, methane sulfonic acid, and sulphonic acid resins. Suitable dibasic acids are ones having 4 to 30 carbon atoms, and include, but are not limited to, succinic acid, glutaric acid, adipic acid, pimelic acid, suberic acid, azelaic acid, sebacic acid, undecanedioic acid, dodecanedioic acid, brassylic acid, phthalic acid, and terephthalic acid. It is also possible to prepare the cyclododecanediol-based polyester polymers and polyester polyols by reacting the cyclododecanediol with the methyl- or ethyl-esters of the dibasic acids rather than the dibasic acids directly. For example, dimethyl terephthalate or diethyl terephthalate can be employed in place of terephthalic acid. Polyesters polymers are useful as engineering resins, fibers, and textiles. Polyester polyols are useful in the production of polyurethanes such as coatings, adhesives, sealants, elastomers, and foams.

What is claimed is:

1. A method for preparing a substantially pure mixture of cyclododecanediol isomers, comprising:
(i) contacting in a vessel cyclododecane and an oxygen-containing gas in the presence of a catalyst to obtain a first mixture comprising cyclododecanediol isomers, cyclododecanol, cyclododecanone, cylclododecanedione, and hydroxy cyclododecanone;
(ii) optionally, isolating cyclododecanol and cyclododecanone;
(iii) distilling the mixture obtained at a temperature between from about 150° C. to about 230° C. to obtain the substantially pure mixture of cyclododecanediol isomers.

2. A method according to claim 1 wherein said mixture consists essentially of 1,4-cyclododecanediol, 1,5-cyclododecanediol, and 1,6-cyclododecanediol.

3. A method according to claim 2 wherein the catalyst is selected from the group consisting of boron compounds including boric acid, cobalt octoate, cobalt naphthenate, cobalt laurate, chromium octoate, chromium naphthenate, chromium laurate, chromium ethylhexanoate, cobalt or chromium salts of fatty acids that are soluble in hydrocarbon solvents, and cobalt or chromium contained on an ion exchange resin.

4. A method according to claim 2, further comprising the step of chemically reducing hydroxy cyclododecanone and cyclododecanedione obtained from the product of step (i), under suitable reduction conditions, to cyclododecanediol.

5. A method according to claim 1 wherein the method further comprises recycling unreacted cyclododecane obtained from step (i) by providing said unreacted cyclododecane to the vessel and repeating step (i).

6. A method of using a substantially pure mixture of cyclododecanediol isomers wherein said method comprises contacting said mixture with ammonia or a carboxylic acid to produce the corresponding diamine or diester.

7. A method according to claim 6 wherein said carboxylic acid is selected from the group consisting of acrylic acid, acetic acid, and propionic acid.

8. A method of using a substantially pure mixture of cyclododecanediol isomers wherein said method comprises contacting said mixture with phenol to produce bis-phenol cyclododecane.

9. A method of using a substantially pure mixture of cyclododecanediol isomers wherein said method comprises contacting said mixture with dibasic acids, methyl esters of dibasic acids or ethyl esters of dibasic acids to produce the corresponding cyclododecanediol-based polyester polymers or polyols.
